(19) United States
(12) Patent Application Publication
(10) Pub. No.: US 2012/0179198 A1
(43) Pub. Date: Jul. 12, 2012

(54) SUTURE WITH FILAMENTS FORMED OF POLYETHER-KETONE VARIANT

(76) Inventors: John W. Schmieding, Naples, FL (US); Tara L. Schaneville, Bonita Springs, FL (US)

(21) Appl. No.: 13/428,893

(22) Filed: Mar. 23, 2012

Related U.S. Application Data
(63) Continuation of application No. 11/775,079, filed on Jul. 9, 2007.
(60) Provisional application No. 60/819,001, filed on Jul. 7, 2006.
(60) Provisional application No. 60/915,296, filed on May 1, 2007.

(51) Int. Cl. A61L 17/04

(52) U.S. Cl. 606/228

(57) ABSTRACT

A high strength abrasion resistant surgical suture material with improved tie down characteristics and tissue compliance with braided yarns formed of ether-ketone variant. The suture features a multifilament jacket formed of braided yarns of ether-ketone variant, optionally braided with yarns of polyester, silk, nylon, ultrahigh molecular weight polyethylene or aramid fibers. The braided jacket surrounds a core formed of twisted yarns of ether-ketone variant or ultrahigh molecular weight polyethylene. The suture has exceptional strength, is ideally suited for most orthopedic procedures, and can be attached to a suture anchor or a curved needle.

### CROSS-REFERENCE TO RELATED APPLICATIONS

This is a continuation of U.S. application Ser. No. 11/775,079, filed on Jul. 9, 2007, which claims priority to U.S. Provisional Application Nos. 60/819,001, filed on Jul. 7, 2006; and 60/915,296, filed on May 1, 2007, the entire disclosures of which are hereby incorporated by reference.

### FIELD OF THE INVENTION

The present invention relates to high strength surgical suture materials, and more particularly, to braided suture blends having a polyether-ketone variant, with coatings to improve handling, and colored yarns for tracing and identifying the suture.

### BACKGROUND OF THE INVENTION

Sutures are categorized into several groups—absorbable and non-absorbable; monofilament and multifilament; natural and synthetic. Absorbable sutures degrade by two major mechanisms: (i) sutures of biological origin such as surgical gut are gradually digested by tissue enzymes; and (ii) sutures manufactured from synthetic polymers are principally broken down by hydrolysis in tissue fluids. Non-absorbable sutures, made from a variety of non biodegradable materials, are ultimately encapsulated or walled off by fibroblasts.

A monofilament suture is made of a single strand. A multifilament suture consists of several filaments twisted or braided together. A multifilament gives good handling and tying qualities. Examples of natural sutures are catgut and silk. Synthetic suture material comprises polyglycolic acid (dexon), polyglactin (vicryl), polydioxone (PDS), polyglyconate (Maxon), polyamide (nylon), polyester (dacron), polypropylene (prolene).

Suture strength is an important consideration in any surgical suture material. One of the strongest materials currently formed into elongated strands is an ultrahigh molecular weight long chain polyethylene (UHMWPE), typically used for fishing line and the like, which is sold under the trade names Dyneema and Spectra. This material is much stronger than ordinary surgical suture, however, it does not have acceptable knot tie down characteristics itself for use in surgical applications. However, an exceptionally strong suture with acceptable handling characteristics can be formed by braiding yarns of UHMWPE with polyester, as disclosed and claimed in U.S. Pat. No. 6,716,234, herein incorporated by reference.

Although not previously recognized in the art, a material which can be used in a high strength suture, either alone or in combination with other materials such as ultrahigh molecular weight polyethylene, is PEEK. PEEK is an abbreviation for polyetherether-ketone, a high performance engineering thermoplastic. It is a semi-crystalline, linear aromatic polymer and is widely regarded as the highest performance thermoplastic material currently available. PEEK has excellent chemical resistance, very low moisture absorption, inherently good wear and abrasion resistance, and is unaffected by continuous exposure to hot water or steam. The advantages of PEEK are described in a white paper entitled, “New Materials in Sports Medicine,” Arthrex, Inc., 2005, the disclosure of which is incorporated by reference. Advantages of PEEK generally known in the art are discussed below. PEEK is sold under the trademark PEEK™ by Victrex PLC; http://www.victrex.com/en/index.php; 3 Caledon Court—Suite A, Greenville, S.C. 29615, USA. PEEK is also sold by Invibio; http://www.invibio.com.

PEEK polymer has excellent friction and wear properties which are optimized in the specially formulated tribological grades 450FC30 and 150FC30. These materials exhibit outstanding wear resistance over wide ranges of pressure, velocity, temperature and counterfacial roughness. PEEK polymer has excellent resistance to a wide range of chemical environments, even at elevated temperatures. PEEK grades offer chemical and water resistance similar to PPS (polyphenylene sulfide), but can operate at higher temperatures. It can be used continuously to 480° F. (250° C.) and in hot water or steam without permanent loss in physical properties. The only common environment which dissolves PEEK polymer is concentrated sulphuric acid, nitric and hydrochloric acid. For hostile environments, PEEK is a high strength alternative to fluoropolymers.

PEEK polymer and compounds are not chemically attacked by water or pressurized steam. Components which are constructed from these materials retain a high level of mechanical properties when continuously conditioned in water at elevated temperatures and pressures.

Other properties of PEEK are included in the following Table:

TABLE 1
Property Table

| Properties | ASTM or Unit | PEEK ™ |
|---|---|---|
| Specific Gravity | D792 | 1.30~1.32 |
| Elongation % | D638 | 20~60 |
| Tensile Strength (psi) | D638 | 14,065~14,500 |
| Flexural Strength (psi) | D790 | 24,650 |
| Compressive Strength | D695 | 17,110 |
| Tensile Elastic Modulus | D638 | 522,000 |
| (Young's Modulus) (psi) | | |
| Flexural Modulus | D790 | 580,000~594,500 |
| (psi) | | 10³ MPa(10³ kgf/m²) |
| Hardness Durometer | D636 | D85~86 |
| Shore D | | |
| Coefficient of Friction | D1984 | 0.18 |
| on steel | | (Dynamic) |
| Impact Strength IZOD | D256 | 1.6 |
| 73° F./23° C., | | |
| notched ft/lbs/in | | |
| Melting Point | ° C. | 340 |
| | (° F.) | (644) |
| Upper Service | ° C. | 260 |
| Temperature(20,000 h) | (° F.) | (500) |
| Flame Rating | UL 94 | V-0 |
| Thermal Conductivity | BTU/hr/ft²/deg F. in | 1.73 |
| Linear Coefficient of | D696 | 2.6 |
| Thermal Expansion | | 10⁻⁵° F.−1 |
| Dielectric Constant | 50 Hz-10 kHz | 3.20~3.30 |
| Dielectric Strength | D149 | >500 |
| 10 mil film | | |
| Volume Resistivity | D257 | 4.9 × 10¹⁶ |
| ohm-cm | | |
| Surface Resistivity | D257 | 2.0 × 10¹⁶ |
| ohm/sq. | | |
| Chemical/Solvent | D543 | Excellent |
| Resistance | | |
| Water Absorption, 24 h, % | D570 | 0.5 |
| Refractive Index | | 2.15 |
| Limiting Oxygen Index % | D2863 | 24 |

It has not been previously contemplated in the art to provide PEEK yarns (made in whole or in part of PEEK yarns) in a suture to improve the handling characteristics and tissue compatibility of a suture.

PEEK has been used widely in aerospace, automotive, electronics, defense, food processing, and medical applications. Such use is the result of the properties of PEEK—chemical resistance; high strength for application/part longevity; inherent purity and extremely inert for sterile environments; outstanding autoclavability; impact and wear resistance; and processing and design flexibility.

High strength sutures incorporating PEEK yarns would add to the surgical arts, particularly in areas of orthopedic surgery. Most beneficial would be high strength sutures with PEEK that manifest acceptable knot tie-down characteristics and handling. Also beneficial would be sutures with PEEK that manifest improved abrasion resistance, high mechanical strength, excellent stress cracking resistance and hydrolytic stability in the presence of hot water, steam, solvents and chemicals.

Other materials which have better material properties than the ordinary surgical suture material are members of the polyether ketone family—polyetherketone (PEK), polyetherketoneketone (PEKK), and other polymer variants of ether and ketone.

The ideal suture for use in surgery would be one that is biologically inert and causes no tissue reaction. To avoid an excess tissue reaction, a surgeon should choose the smallest diameter suture with sufficient strength for the task at hand. Further, the suture must be easy for the surgeon to handle and knot reliably. The suture must have stiffness substantially similar to bone. Accordingly, the need exists for a suture material that provides high strength, is easy to handle and to knot reliably, and causes no tissue reaction.

### SUMMARY OF THE INVENTION

The present invention overcomes the disadvantages of the prior art and fulfills the needs noted above by providing high strength suture materials, particularly, braided suture blends formed with polyether ketone variants having surgically-useful qualities, including knot tie down characteristics and handling.

The suture features PEEK yarns (made in whole or in part of PEEK yarns), optionally blended with UHMWPE for strength or enhancement fibers to improve handling characteristics and tissue compatibility, for example, of the high strength suture material. Yarns of this material are much stronger than those used to make ordinary surgical suture. Enhancements in tissue compatibility include improving compliance by allowing the ends of the suture to be cut close to the knot without concern for deleterious interaction between the ends of the suture and surrounding tissue. Other enhancements include incorporating visible traces into the finished suture.

The high strength sutures of the present invention preferably are formed by braiding. Plain hollow braids of PEEK are most preferred, though the various other types of braiding can be used. One or more enhancement fibers or yarns can be blended into the braid. The sutures also can include a core, preferably formed of twisted yarns. In a preferred embodiment, the core includes, or is made exclusively of, PEEK. Other core materials can be used in place of or in addition to PEEK, for example, ultrahigh molecular weight polyethylene.

In another embodiment, the suture features a jacket made of a blend of ultrahigh molecular weight polymer yarns, for example, ultrahigh molecular weight polyethylene, and one or more thermoplastic fiber, preferably a member of the polyether-ketone family. The UHMWPE provides strength. The polyether-ketone provides improved mechanical properties including tie down properties and improved abrasion resistance. Handling properties of the high strength suture may be enhanced using various materials to coat the suture.

As a further enhancement, yarns of a contrasting color may be added to the braided threads to enhance visibility and to make the suture more discernable during surgical procedures. The colored yarns preferably are dyed. Natural fibers, such as silk, and some synthetic fibers, accept dye more readily than others. Other synthetic fibers can be colored during manufacture by tinting the polymeric material from which they are formed. In a further aspect of the invention, colored traces can be produced by exposing the braided suture material to a dye that is accepted by some strand materials and rejected by others. Those yarns that accept the dye become the colored trace, while yarns that reject the dye remain their original color, such as translucent or white.

In one embodiment, half of a length of suture is provided with tinted tracing yarns, or otherwise contrasts visually with the other half of the length of suture, which remains a plain, solid color, or displays a different tracing pattern, for example. Accordingly, when the length of suture is loaded through the eyelet of a suture anchor or passed through tissue, for example, at least one of the legs of the suture is visually coded, making identification and handling of the suture legs simpler. A few trace threads having a contrasting color, preferably of a readily dyed yarn such as polyester or nylon, in the cover aid surgeons in identifying the travel direction of the suture during surgery, particularly during arthroscopic operations and others, such as endoscopy and laparoscopy, that currently are generally referred to as “minimally invasive.” Providing the trace threads in a regularly repeating pattern is particularly useful, allowing the surgeon to distinguish different ends of lengths of suture, and determine the direction of travel of a moving length of suture. Of the more easily dyed yarns, nylon is preferred in that it accepts dye readily.

In a preferred embodiment, the suture includes a multifilament jacket or sheath formed of braided PEEK. Optionally, the PEEK can be braided with an enhancement fiber or yarn from the group consisting of polyester, silk, nylon, ultrahigh molecular weight polyethylene and aramid, and combinations thereof. The jacket surrounds a core made substantially or entirely of PEEK. The core preferably includes three yarns of PEEK (100 decitex) twisted at about three to six twists per inch.

The jacket most preferably comprises eight yarns of PEEK (94 decitex yarn with 30 filaments) braided with eight (8) yarns of polyester (95 decitex). Optionally, one or more yarns of a material that can be dyed, such as nylon, can be provided in black or some other contrasting color as explained in greater detail below.

In another embodiment, the suture includes a multifilament jacket formed of ultrahigh molecular weight polyethylene yarn braided with PEEK. The jacket surrounds a core substantially or entirely of ultrahigh molecular weight polyethylene. The core preferably includes three (3) yarns of ultrahigh molecular weight polyethylene (144 decitex), twisted at about three to six twists per inch.

The jacket preferably includes eight (8) yarns of ultrahigh molecular weight polyethylene (144 decitex) braided with eight (8) yarns of PEEK (100 decitex yarn with 30 filaments). Optionally, one or more yarns such as nylon may be provided in black or some other contrasting color as explained in greater detail below.

The suture of the present invention advantageously has the strength of Ethibond No. 5 suture, yet has the diameter, feel, and tie-ability of No. 2 suture. As a result, the suture of the present invention is ideal for most orthopedic procedures such as rotator cuff repair, Achilles tendon repair, patellar tendon repair, ACL/PCL reconstruction, hip and shoulder reconstruction procedures, and replacement for suture used in or with suture anchors.

The suture can be uncoated or coated. Typically useful coatings include wax (beeswax, petroleum wax, polyethylene wax, or others), silicone (Dow Corning silicone fluid 202A or others), silicone rubbers (Nusil Med 2245, Nusil Med 2174 with a bonding catalyst, or others), PTFE (Teflon, Hostaflon, or others), PBA (polybutylate acid), ethyl cellulose (Filodel), and others known in the art. The coatings improve lubricity of the braid, and thus improve the handling characteristics, such as knot security, or abrasion resistance, for example.

As an added advantage, as mentioned above, some of the yarns in the cover may be provided in a contrasting color for visibility and identification purposes. A few trace threads having a contrasting color, preferably of a readily dyed yarn such as polyester or nylon, in the cover aid surgeons in identifying the travel direction of the suture during surgery, particularly during arthroscopic operations. Providing the trace threads in a regularly repeating pattern is particularly useful, allowing the surgeon to distinguish different ends of lengths of suture, and determine the direction of travel of a moving length of suture. Of the more easily dyed yarns, nylon is preferred in that it accepts dye readily.

Other features and advantages of the present invention will become apparent from the following description of the invention which refers to the accompanying drawings.

### BRIEF DESCRIPTION OF THE DRAWINGS

FIG. 1 is an enlarged detail view of a section of suture according to the present invention.

FIG. 2 is a schematic cross section of a length of suture according to the present invention.

FIG. 3 is an illustration of the suture of the present invention attached to a suture anchor loaded onto a driver.

FIGS. 4A and 4B show the suture of the present invention attached to a half round, tapered needle.

FIG. 5 illustrates a bulk length of suture of the present invention.

FIG. 6 illustrates a strand of suture according to the present invention provided on a suture anchor.

### DETAILED DESCRIPTION OF THE INVENTION

The present invention is a high strength surgical suture material with surgically-useful qualities, including knot tie down characteristics and handling.

For purposes of simplicity and clarity, the term “PEEK,” as used herein, is to be understood as including all variants of ether and ketone, including but not limited to, polyetherketone (PEK), polyetherketoneketone (PEKK), polyetherether-ketone (PEEK), and other ether and ketone variants.

The term “enhancement fiber,” as used herein, is to be understood as including polyester, silk, nylon, ultrahigh molecular weight polyethylene and aramid, and combinations thereof.

The term “yarn(s),” as used herein, is to be understood as including fiber(s), filament(s), and the like used to make a suture of the present invention. Typically, though, yarns are comprised of fibers and/or filaments.

The PEEK component of the present invention provides strength, and the enhancement fiber is provided to improve tie ability and tie down characteristics.

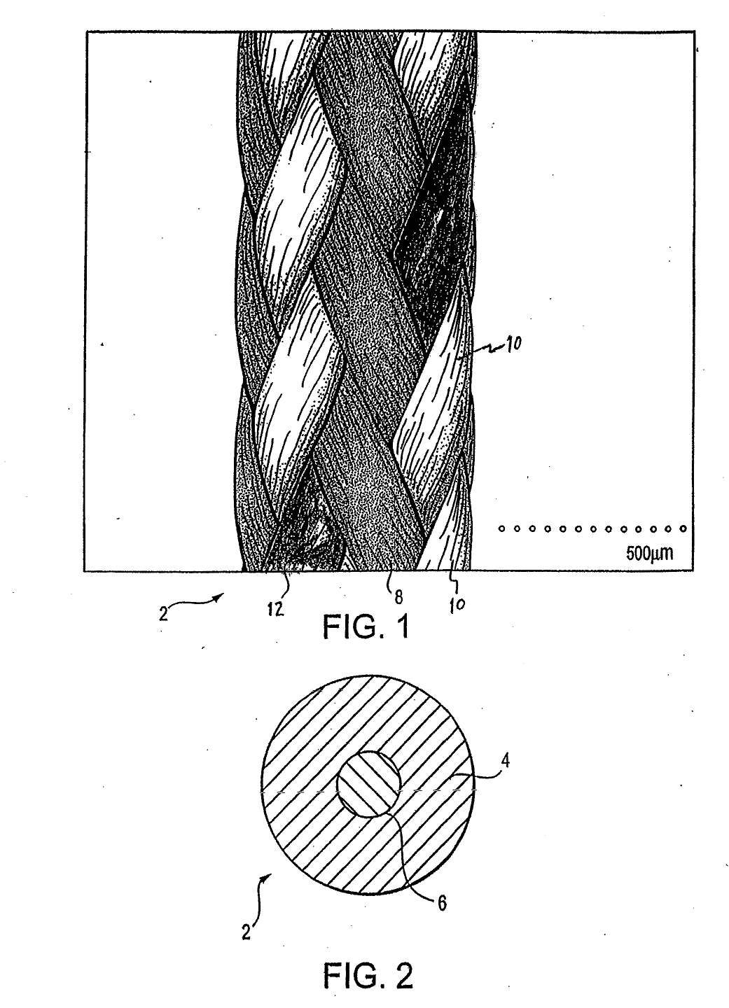

Referring now to the drawings, where like elements are designated by like reference numerals, FIGS. 1 and 2 illustrate a section of suture 2 according to the present invention shown enlarged several fold. As illustrated in FIG. 2, suture 2 is made up of a jacket 4 and a core 6 surrounded by the jacket 4. Yarns of PEEK 8, optional yarns of enhancement fiber 10, and optional colored yarns 12 are braided together to form the jacket 4. Core 6 is formed of twisted yarns of PEEK or ultrahigh molecular weight polyethylene.

In accordance with the present invention, optional colored traces 12 are preferably black. The black trace assists surgeons in distinguishing between suture lengths with the trace and suture lengths without the trace. Traces also assist the surgeon in identifying whether or not, and in what direction, the suture is moving. The trace can extend the entire length of the suture or only on half of a length of suture, the other half of the suture length remaining plain (white). Alternatively, the traces can form visibly distinct coding patterns on each half of the suture length. As a result, when the suture is threaded through the eyelet of a suture anchor, for example, the two legs (halves) of the length of suture are easily distinguished, and their direction of travel will be readily evident when the suture is pulled during surgery. Other patterns and arrangements of tracings also can be provided.

Ultrahigh molecular weight polyethylene yarns 8 are substantially translucent or colorless. All or the majority of the PEEK yarns 10 are white (undyed). Optionally, one or more PEEK or nylon yarns 12 may be provided in a contrasting color provide a trace in the suture. Due to the transparent nature of the ultrahigh molecular weight polyethylene, the suture takes on the color of yarns 10 and 12, and thus appears to be white with a trace in the contrasting color. In accordance with the present invention, trace yarns 12 are preferably provided in black. The black trace assists the surgeon in differentiating between suture strands with the trace and suture strands without the trace. The trace also assists the surgeon in identifying whether the suture is moving.

The colored yarns preferably are dyed. Natural fibers, such as silk, and some synthetic fibers, accept dye more readily than others. Other synthetic fibers can be colored during manufacture by tinting the polymeric material from which they are formed. In a further aspect of the invention, colored traces can be produced by exposing the braided suture material to a dye that is accepted by some materials and rejected by others. Those yarns that accept the dye become the colored trace, while yarns that reject the dye remain their original color, such as translucent or white.

In one embodiment, half of a length of suture is provided with tinted tracing yarns, or otherwise contrasts visually with the other half of the length of suture, which remains a plain, solid color, or displays a different tracing pattern, for example. Accordingly, when the length of suture is loaded through the eyelet of a suture anchor or passed through tissue, for example, at least one of the legs of the suture is visually coded, making identification and handling of the suture legs simpler. Easy identification of suture in situ is advantageous in surgical procedures, particularly during arthroscopic surgeries and others, such as endoscopy and laparoscopy, that currently are generally referred to as “minimally invasive.”

Details of the present invention will be described further below in connection with the following examples:

### Example 1

Core: 3 twisted yarns of ultrahigh molecular weight polyethylene (144 decitex each)

Jacket: 8 yarns PEEK (100 or 94 decitex) braided with 8 yarns ultrahigh molecular weight polyethylene (144 decitex)

The suture includes a multifilament jacket formed of ultrahigh molecular weight polyethylene yarn braided with PEEK. The jacket surrounds a yarn core substantially or entirely of ultrahigh molecular weight polyethylene.

The jacket is formed using eight yarns of 100 or 94 decitex PEEK braided with eight yarns of 144 decitex ultrahigh molecular weight polyethylene. The core is formed of three twisted yarns of 144 decitex ultrahigh molecular weight polyethylene, twisted at about three to six twists per inch.

### Example 2

Core: 1 yarn of ultrahigh molecular weight polyethylene (144 decitex)

Jacket: 8 twisted yarns PEEK (each yarn made of 2 twisted yarns of 45 decitex) braided with 8 yarns ultrahigh molecular weight polyethylene (144 decitex)

The jacket is formed using eight twisted yarns of PEEK braided, each yarn comprised of two twisted yarns of 45 decitex each, With eight yarns of 144 decitex ultrahigh molecular weight polyethylene. The core is formed of a yarn of 144 decitex ultrahigh molecular weight polyethylene.

### Example 3

Core: 3 yarns of PEEK (100 decitex each)

Jacket: 8 twisted yarns PEEK (94 or 100 decitex) braided with 8 yarns ultrahigh molecular weight polyethylene (144 decitex)

The jacket is formed using eight yarns of 94 or 100 decitex PEEK braided with eight yarns of 144 decitex ultrahigh molecular weight polyethylene. The core is formed of three twisted yarns of 100 decitex PEEK.

### Example 4

Core: 3 twisted yarns of ultrahigh molecular weight polyethylene (144 decitex each)

Jacket: 8 twisted yarns PEEK (90 decitex) braided with 8 yarns ultrahigh molecular weight polyethylene (144 decitex)

The core is formed using three twisted yarns of 144 decitex ultrahigh molecular weight polyethylene, twisted at about three to six twists per inch. The jacket is formed using eight yarns of 144 decitex ultrahigh molecular weight polyethylene, braided with eight twisted yarns of PEEK.

### Example 5

Core: 3 twisted yarns of ultrahigh molecular weight polyethylene (144 decitex each)

Jacket: 16 yarns PEEK (94 or 100 decitex)

The jacket is formed using sixteen yarns of 94 or 100 decitex PEEK. The core is formed of three twisted yarns of 144 decitex ultrahigh molecular weight polyethylene.

### Example 6

Core: 3 twisted yarns of PEEK (100 decitex each)

Jacket: 16 yarns PEEK (94 or 100 decitex)

The jacket is formed using sixteen yarns of 94 or 100 decitex PEEK. The core is formed of three twisted yarns of 100 decitex PEEK.

### Example 7

### USP Size 5 (EP size 7)

Made on a 16 carrier Hobourns machine, the yarns used in the hollow, plain braided jacket are PEEK, polyester type 712, and nylon. The jacket is formed using eight (8) yarns of PEEK per carrier, braided with six (6) yarns of 190 decitex polyester, and two (2) yarns of tinted nylon. The core is formed of three (3) carriers of PEEK or ultrahigh molecular weight polyethylene braided at three (3) to six (6) twists per inch. A No. 5 suture is produced.

### Example 8

### Silk—Size 1

Core: 1 end of 100 decitex PEEK×3 ply

Jacket: 5 carriers 95 decitex polyester; 6 carriers 100 decitex PEEK; 1 carrier 84 decitex silk

The jacket is formed using six yarns of 100 decitex PEEK, braided with five yarns of 95 decitex polyester, and one strand of 84 decitex silk. The core is formed of three twisted yarns of 100 decitex PEEK or 144 decitex ultrahigh molecular weight polyethylene.

### Example 9

### Silk—Size 2

Core: 1 end of 100 decitex PEEK×3 ply

Jacket: 7 carriers 95 decitex polyester; 8 carriers 100 decitex PEEK; 1 carrier 84 decitex silk

The jacket is formed using eight yarns of 100 decitex PEEK, braided with seven yarns of 95 decitex polyester, and one yarn strand of 84 decitex silk. The core is formed of three twisted yarns of 100 decitex PEEK or 144 decitex ultrahigh molecular weight polyethylene.

### Example 10

Core: 3 yarns of ultrahigh molecular weight polyethylene (144 decitex)

Jacket: 8 yarns of 144 decitex ultrahigh molecular weight polyethylene; 8 yarns of PEEK (94 or 100 decitex)

The core is formed using three yarns of 144 decitex ultrahigh molecular weight polyethylene, twisted at about three to six twists per inch. The jacket is formed using eight yarns of 144 decitex ultrahigh molecular weight polyethylene, braided with eight yarns of 94 or 100 decitex PEEK with thirty yarns.

As stated previously, one or more yarns in the blend of any of the above examples can be provided in pre-dyed colors, e.g., black, to provide a trace. The trace threads enhance the ability to visually detect suture motion and the ability to differentiate between colored and uncolored suture strands.

To make various sizes of the inventive suture, different decitex values and different PPI settings can be used to achieve the required size and strength needed. In addition, smaller sizes may require manufacture on 12, 8 or 6 carrier machines, for example. The very smallest sizes can be made without a core. Overall, the suture may range from 5% to 90% PEEK (preferably at least 31% of the yarns are PEEK), with the balance formed of enhancement fibers, such as polyester and/or silk. The core preferably comprises 12.6% or greater of the total amount of yarn.

The suture preferably is coated with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the braid, knot security, or abrasion resistance, for example.

According to an alternative embodiment of the present invention, a partially bioabsorbable suture is provided by blending PEEK yarns with a bioabsorbable material, such as PLLA or one of the other polylactides, for example. A suture made with about 10% PEEK blended with absorbable yarns would provide greater strength than existing bioabsorbable suture, and with less stretch. Over time, 90% or more of the suture would absorb, leaving only a very small remnant of the knot. The absorbable suture can include coatings and tinted traces as noted above for nonabsorbable suture.

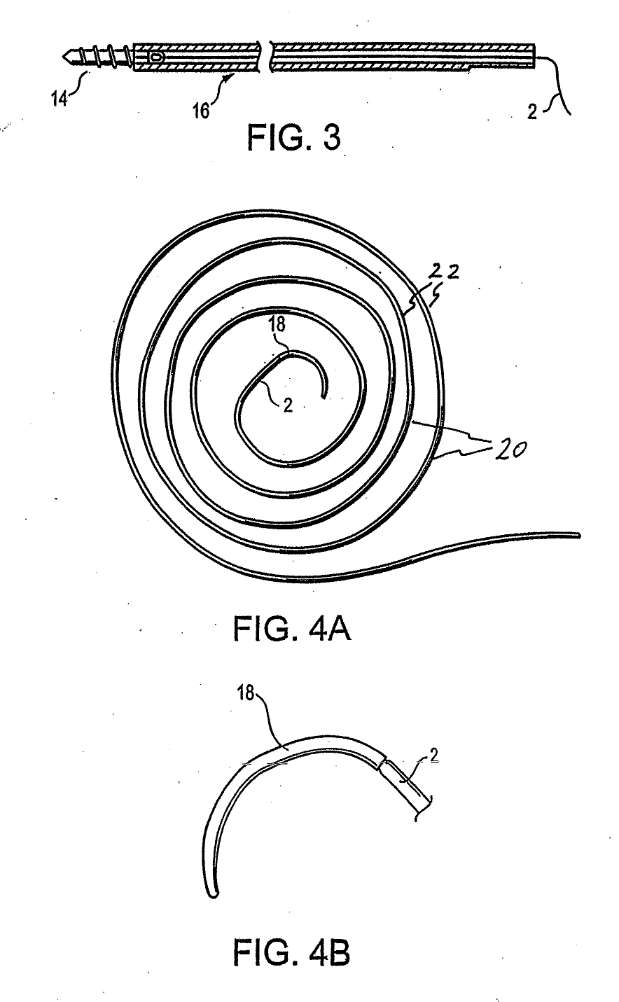

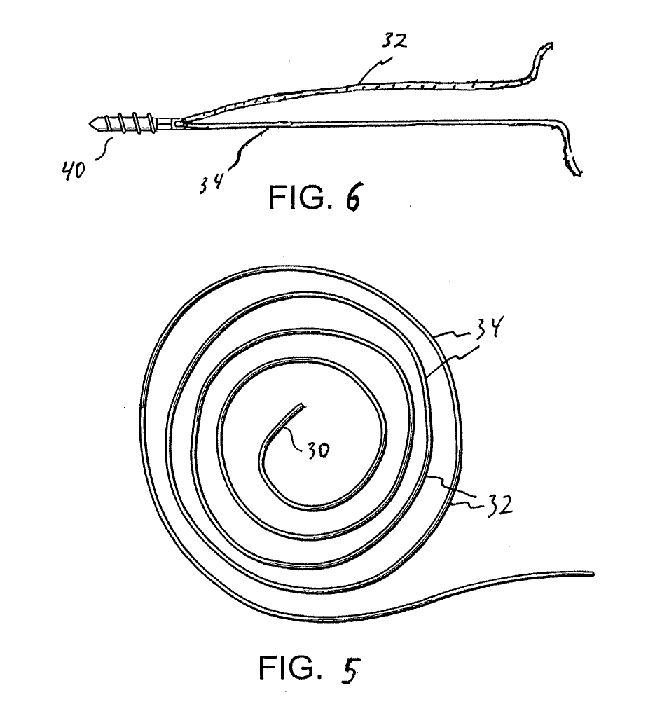

In one method of using the suture of the present invention, the suture 2 is attached to a suture anchor 14 as shown in FIG. 3 (prepackaged sterile with an inserter 16), or is attached at one or both ends to a half round, tapered needle 18 as shown in FIGS. 4A and 4B, or to a straight needle (not shown). FIG. 4A also illustrates a length of suture having regularly repeating pattern of trace threads according to the present invention. Sections 20 of the length of suture 2 have tinted tracing threads woven in, while sections 22 of the length of suture are plain, or otherwise are distinguishable from sections 20. The alternating patterned and plain sections aid the surgeon in determining the direction of suture travel when pulling the suture through tissue as viewed through an arthroscope, for example.

In yet another embodiment, as shown in FIG. 5, to make the suture which has a trace only at one end, bulk suture 30 is provided with repeating sections 32 having trace threads separated by sections 34 having no trace threads. The bulk suture is cut between every other section, at one end of each plain section, for example, to provide lengths of suture that are half traced and half plain. Alternatively, the bulk suture can be cut midway through each section to provide a shorter suture having a trace at one end.

In yet another embodiment, the half-and-half lengths of suture can be threaded through the eyelet of a suture anchor 40, as shown in FIG. 6. As a further alternative, uniform lengths of the braided suture can be exposed, partially or completely, to the dye (dipped, sprayed, etc.) to provide suture lengths with partial or complete dying patterns. Accordingly, the identity of each leg of the suture strand provided on the suture anchor is easily decoded by a surgeon operating with the suture anchor assembly.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A suture strand comprising a plurality of braided yarns formed of a polyether-ketone variant.

2. The suture strand of claim 1, further comprising a core comprising yarns of polyether-ketone variant surrounded by a jacket comprising yarns of polyether-ketone variant.

3. The suture strand of claim 1, wherein the yarns of polyether-ketone variant comprises at least 31% of the yarns in the suture strand.

4. The suture strand of claim 1, wherein the core comprises about 12.6% or greater of the total amount of yarns in the suture strand.

5. The suture strand of claim 2, wherein the core comprises braided yarns of polyether-ketone variant.

6. The suture strand of claim 1, wherein the polyether-ketone variant is selected from a group consisting of polyetherketone, polyetherketoneketone, and polyetherether-ketone.

7. The suture strand of claim 1, further comprising a coating disposed on the jacket, the coating being selected from the group consisting of wax, silicone, silicone rubbers, PTFE, PBA, and ethyl cellulose.

8. The suture strand of claim 1, wherein the core is formed of at least three yarns of the polyether-ketone variant twisted at three to six twists per inch.

9. The suture strand of claim 1, wherein the jacket is formed of yarns of the polyether-ketone variant braided with yarns of polyester.

10. The suture strand of claim 1, wherein the jacket is formed of yarns of the polyether-ketone variant braided with yarns of ultrahigh molecular weight polyethylene.

11. The suture strand of claim 1, wherein further comprising a core formed of twisted yarns of the polyether-ketone variant.

12. The suture strand of claim 1, wherein further comprising a core formed of twisted yarns of ultrahigh molecular weight polyethylene.

13. A suture assembly comprising:
a suture, the suture having a longitudinal length and a multifilament jacket comprising a plurality of braided yarns of polyether-ketone variant; and
a suture anchor, wherein the suture is threaded through an eyelet of the suture anchor.

14. A method of using a suture strand, comprising the steps of:
cutting a bulk length of multifilament suture material to make a plurality of suture yarns, the multifilament suture material comprising a plurality of braided yarns of polyether-ketone variant; and
attaching one of the plurality of suture yarns to a suture anchor by threading through an eyelet of the suture anchor.
